(19) United States
(12) Patent Application Publication
(10) Pub. No.: US 2022/0008311 A1
(43) Pub. Date: Jan. 13, 2022

(54) TWO-COMPONENT SYSTEM FOR SMOOTHING AND CARE OF HAIR

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE); ELISABETH POPPE, Hamburg (DE)

(73) Assignee: Henkel AG & Co. KGaA, Duesseldorf (DE)

(21) Appl. No.: 17/289,586

(22) PCT Filed: Oct. 31, 2019
(86) PCT No.: PCT/EP2019/079787

§ 371 Date: Apr. 28, 2021

(30) Foreign Application Priority Data
Oct. 31, 2018 (DE) 10 2018 127 187.5

(51) Int. Cl. A61K 8/58; A61K 8/31; A61K 8/34; A61K 8/19; A61K 8/41; A61K 8/39; A61Q 5/04

(52) U.S. Cl. CPC A61K 8/585; A61K 8/31; A61K 8/342; A61Q 5/04; A61K 8/19; A61K 8/416; A61K 8/39; A61K 8/34

(57) ABSTRACT

The present disclosure relates to a two-component system for straightening and care of keratinous material, wherein the two-component system separately comprising an anhydrous carrier medium that comprises an alkane, a fatty alcohol, and an alcohol, and an aqueous phase, which in turn contains an alkali metal hydroxide. The present disclosure further relates to the use of the two-component system for straightening hair.

### CROSS-REFERENCE TO RELATED APPLICATION

This application is a U.S. National-Stage entry under 35 U.S.C. § 371 based on International Application No. PCT/EP2019/079787, filed Oct. 31, 2019, which was published under PCT Article 21(2) and which claims priority to German Application No. 102018127187.5, filed Oct. 31, 2018, which are all hereby incorporated in their entirety by reference.

### TECHNICAL FIELD

The present disclosure relates to a two-component system for straightening and care of keratinous material, wherein the two-component system, separated from each other contains an anhydrous carrier medium that comprises an alkane, a fatty alcohol, and an alcohol, and an aqueous phase which in turn contains an alkali metal hydroxide. The present disclosure further relates to the use of the two-component system for straightening hair.

### BACKGROUND

External stressing of the hair due to chemical materials from a variety of different sources poses challenges for the development of cosmetic care products. Furthermore, frequently changing customer requests regarding a certain finish of the hair are linked to a recurrent chemical stress on the hair. For example, hair coloring or hair straightening stress the hair, due to which, an intensive care may be necessary.

Air and water pollution adversely affects skin and hair. The presence of other air pollutants and to UV radiation can reinforce the effect of various air pollutants. It is known that the toxicity of gaseous pollutants of the air, such as sulfur dioxide, ozone and nitrogen oxides, especially depends on their initiator activity for free radicals, which causes damages in living beings. Free radicals are metabolic products that also occur naturally in the body. Such cases are referred to as oxidative damage. Free radicals can also cause hair damage, evidenced, for example, by a reduction in the shine as well as of the grip and/or fading of the hair color.

Developers of hair products face a two-fold challenge, satisfying customer demands regarding hair shape, color and finish poses a challenge on one hand and preserving the structure, especially the surface structure of the hair, on the other. Hair straightening and hair coloring have proved especially difficult to manage. The problem of a damaging attack on the hair is worsened by the damaging environmental influences mentioned above.

Organosilicon compounds from the group of silanes are described in the state of the art, which comprise at least one hydroxy group and/or hydrolysable group. Due to the presence of hydroxy groups and/or hydrolysable groups, silanes are reactive substances that hydrolyze or oligomerize or polymerize in the presence of water. When used on a keratinous material, oligomerization or polymerization of the silanes initiated by the presence of water eventually causes the formation of a film that can provide protection.

### BRIEF SUMMARY

Products for the straightening and care of keratinous material are provided, and methods of using the same. In an exemplary embodiment, a product includes a two-component system with an anhydrous carrier medium as a first component and an aqueous phase as a second component. The anhydrous carrier medium comprises a branched or linear C8-C30 alkane, a branched or linear C10-C30 fatty alcohol, and a branched or linear monovalent C2-C8 alcohol. The aqueous phase comprises water and an alkali metal hydroxide.

A method of using the product for straightening and care of keratinous material is provided in another embodiment. The method includes applying a two-component system to the keratinous material, where the two-component system includes an anhydrous carrier medium as a first component and an aqueous phase as a second component. The anhydrous carrier medium comprises a branched or linear C8-C30 alkane, a branched or linear C10-C30 fatty alcohol, and a branched or linear monovalent C2-C8 alcohol. The aqueous phase comprises water and an alkali metal hydroxide.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The problem in using organosilicon compounds is their instability against water. Aqueous systems for hair treatment are thus disadvantageous if they contain organic silicon compounds as active ingredients.

The task underlying the present disclosure includes providing a system that serves as the basis for the manufacture of a product that makes gentle hair treatment, especially hair straightening, possible. In particular, the product to be manufactured should preserve or restructure the surface structure of hair on using it.

This task is achieved by employing a two-component system for straightening and care of keratinous material, especially hair, wherein the two-component system separately comprises:
An anhydrous carrier medium as a first component, comprising
   a) at least one branched or linear C8-C30 alkane,
   b) at least one branched or linear C10-C30 fatty alcohol, and
   c) at least one branched or linear monovalent C2-C8 alcohol, and
An aqueous phase as a second component, comprising water and an alkali metal hydroxide.

The advantage of the two-component systems lies in providing the option of separately storing the substances that react with each other. Just shortly before using the two-component system, the components can be combined with each other to provide a product that is ready for use. In the context of the present disclosure, thus, the feature “comprise separately” is to be so understood that the two components, namely the anhydrous carrier medium and the aqueous phase, are present in two compartments or two separate containers spatially separated from each other, such that they do not inadvertently come to be mixed.

According to a preferred embodiment of the present disclosure, the carrier medium serves as the basis for the provision of an organosilicon compound. The organic silicon compound remains lab-stable in the carrier medium. The carrier medium can be combined with one or more aqueous phases as the second component, which contains the active ingredients serving for the hair straightening and is water-based. Because the carrier medium is then added to one or the other aqueous phases just shortly before the application, the active ingredient organosilicon compound remains stable until the application.

In the context of the present disclosure, “anhydrous” is to be preferably understood to mean that water is not added to the aqueous carrier medium or the aqueous carrier medium is not water-based. Preferably, the water content of the anhydrous carrier medium is less than 5 weight %, more preferably lower than 2 weight %, most preferably lower than 1 weight %, based on the total weight of the anhydrous carrier medium. If the fluid quantity is low, a small part of the organic silicon compound can hydrolyze and the hydrolysate remains in balance with the free water. This quantity of water is preferably in the above-mentioned quantities.

Keratinous material includes hair, skin, and nails (such as fingernails and/or toenails). Wool, furs and feathers also fall under the definition of keratinous material.

Preferably, a keratinous material is understood to be human hair, human skin and human nails, especially fingernails and toenails. Especially preferably, keratinous material is understood to be human hair, head and beard hair in particular.

According to a preferred embodiment of the present disclosure, the anhydrous carrier medium of the two-component system contains at least one organic silicon compound, namely the one or the multiple compounds which are to be stabilized. Preferred organic silicon compounds are selected from silanes with one, two or three silicon atoms, where the organic silicon compound comprises one or more hydroxyl groups and/or hydrolyzable groups per molecule. By adding organic silicon compounds in the anhydrous carrier, the organic silicon compounds are protected from hydrolysis.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds are compounds containing one to three silicon atoms. Organic silicon compounds preferably contain one or two silicon atoms.

The effect of the organic silicon compounds relates to the protection and the care of the hair, especially the hair surface, upon using the two-component system for straightening of hair. The hydrolyzed silicon compounds, when brought together with the aqueous phase, form a protective film on the hair surface and thus have a “repair” effect.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The anhydrous carrier medium of the two-component system contains, according to the preferred embodiment, at least one organic silicon compound preferably selected from silanes having one, two or three silicon atoms, wherein the organic silicon compound comprises one or more hydroxyl groups or hydrolyzable groups per molecule.

In the context of a particularly preferred embodiment, the anhydrous carrier medium of the two-component system features at least one organic silicon compound which is selected from silanes having one, two or three silicon atoms, wherein the organic silicon compound further comprises one or multiple basic groups and one or multiple hydroxyl groups or hydrolyzable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a di(C₁-C₆)alkylamino group.

The hydrolyzable group(s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R′″Si—O—CH₂—CH₃. The residues R′, R″ and R′″ represent the three remaining free valences of the silicon atom.

Quite particularly good results could be obtained if the anhydrous carrier medium of the two-component system contains at least one organic silicon compound of formula (I) and/or (II).

The compounds of formulas (I) and (II) are organic silicon compounds selected from silanes having one, two or three silicon atoms, the organic silicon compound comprising one or more hydroxyl groups and/or hydrolysable groups per molecule.

In another quite particularly preferred embodiment, the anhydrous carrier medium of the two-component system contains at least one organic silicon compound of formula (I) and/or (II).

R₁R₂N-L-Si(OR₃)ₐ(R₄)_{b} (I),

where
R₁, R₂ both represent a hydrogen atom, and
L represents a linear, divalent C₁-C₆-alkylene group, preferably a propylene group (—CH₂—CH₂—CH₂—) or an ethylene group (—CH₂—CH₂—),
R₃, R₄ independently represent a methyl group or an ethyl group,
a stands for the number 3 and
b stands for the number 0,

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5, R5′, R5″ independently represent a hydrogen atom or a C₁-C₆ alkyl group,
R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g and h is different from 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl. Preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, and the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH₂—CH₂—CH₂—) and the butylene group (—CH₂—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, divalent C₁-C₂₀ alkylene group.

Preferably -L- stands for a linear, divalent C₁-C₂₀ alkylene group. Further preferably -L- stands for a linear divalent C₁-C₆ alkylene group. Particularly preferred -L stands for a methylene group (CH₂—), an ethylene group (—CH₂—CH₂—), a propylene group (—CH₂—CH₂—CH₂—) or a butylene group (—CH₂—CH₂—CH₂—CH₂—). In particular, L stands for a propylene group (—CH₂—CH₂—CH₂—).

The organic silicon compounds of formula (I)

R₁R₂N-L-Si(OR₃)ₐ(R₄)_{b} (I),

carry the silicon-containing grouping —Si(OR₃)ₐ(R₄)_{b} at one end.

In the terminal structural unit —Si(OR₃)ₐ(R₄)_{b}, R₃ is hydrogen or a C₁-C₆ alkyl group, and R₄ is a C₁-C₆ alkyl group. Particularly preferred, R₃ and R₄ independently of each other represent a methyl group or an ethyl group.

Here a stands for an integer from 1 to 3, and b stands for the integer 3−a. If a stands for the number 3, then b is equal to 0. If a stands for the number 2, then b is equal to 1. If a stands for the number 1, then b is equal to 2.

The best care for stressed hair could be obtained if the anhydrous carrier medium contains at least one organic silicon compound corresponding to formula (I) or formula (II): in which R₃, R₄ independently of each other represent a methyl group or an ethyl group.

Especially well-suited organic silicon compounds of the formula (I) are:
(3-Aminopropyl)triethoxysilane

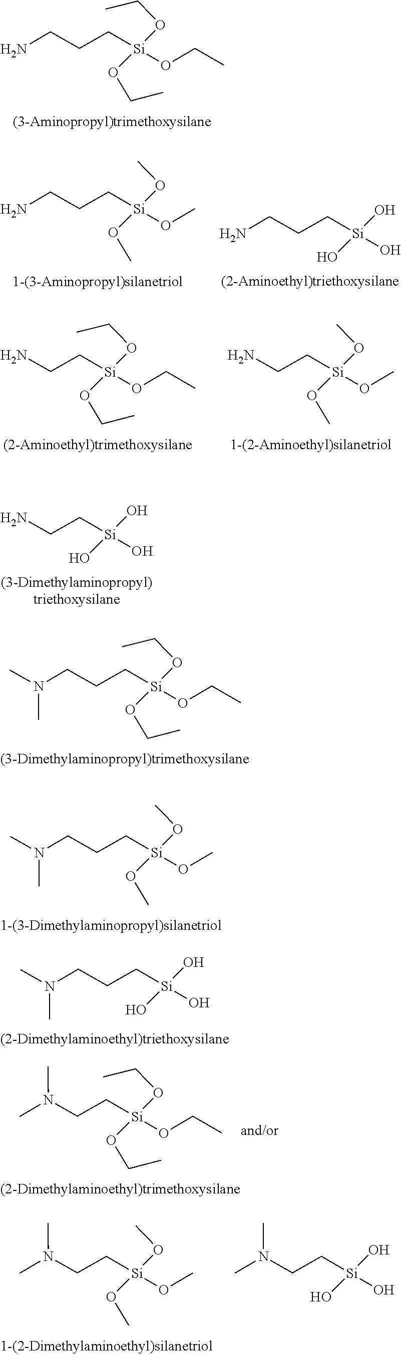

The aforementioned organic silicon compound of formula (I) is commercially available. (3-aminopropyl)trimethoxysilane, for example, can be purchased from Sigma-Aldrich®. (3-aminopropyl)triethoxysilane is also commercially available from Sigma-Aldrich®.

In a further embodiment, the anhydrous carrier medium contains at least one organic silicon compound of formula (II)

(R₅O)_{c}(R₆)_{d}Si-(A)_{c}-[NR₇-(A′)]_{f}-[O-(A″)]_{g}-[NR₈-(A′″)]ₕ—Si(R₆′)_{d′}(OR₅′)_{c′} (II).

The organosilicon compounds of formula (II) each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}-

and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) contains at least one grouping from the group including -(A)- and —[NR₇-(A′)]- and —[O-(A″)]- and —[NR₈-(A′″)]-

In the two terminal structural units (R₅O)_{c}(R₆)_{d}Si— and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here c stands for an integer from 1 to 3, and d stands for the integer 3−c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3−c′. If c′ stands for the number 3, then d′ is 0. If c′ stands for the number 2, then d′ is 1. If c′ stands for the number 1, then d′ is 2.

Very preferred carrier media contain an organic silicon compound, in which the radicals c and c′ both stand for the number 3. In this case d and d′ both stand for the number 0.

In a further preferred embodiment, the anhydrous carrier medium of the two-component system contains at least one organic silicon compound of formula (II)

(R₅O)_{c}(R₆)_{d}Si-(A)ₑ-[NR₇-(A′)]_{f}—[O-(A″)]_{g}-[NR₈-(A′″)]ₕ—Si(R₆′)_{d}′(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the present disclosure corresponds to formula (IIa)

(R₅O)₃Si-(A)_{c}-[NR₇-(A′)]_{f}—[O-(A″)]_{g}-[NR₈-(A′″)]ₕ—Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)e- and -[NR7-(A′)]f- and —[O-(A″)]g- and -[NR8-(A′″)]h- are located in the middle part of the organic silicon compound of formula (II).

In this context, the presence of certain groupings has proved to be particularly beneficial in terms of increasing washability. Particularly good results were obtained when at least two of the residues e, f, g and h stand for the number 1. Especially preferred are when e and f both stand for the number 1. Furthermore, g and h both stand for the number 0.

If e and f both stand for the number 1 and g and h both stand for the number 0, the organic silicon compounds correspond to formula (IIb)

(R₅O)_{c}(R₆)_{d}Si-(A)-[NR₇-(A′)]-Si(R₆′)_{d}′(OR₅′)_{c′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

If the radical f represents the number 1, then the organic silicon compound of formula (II) contains a structural grouping —[NR₇-(A′)]-.

If the radical h represents the number 1, then the organic silicon compound of formula (II) contains a structural grouping —[NR₈-(A′″)]-.

Herein, the radicals R₇ and R₈ independently represent a hydrogen atom, a C₁-C₆ alkyl group, a hydroxy C₁-C₆-alkyl group, a C₂-C₆ alkenyl group, an amino-C₁-C₆-alkyl group or a grouping of the formula (III)

(A″″)-Si(R₆″)_{d}″(OR₅″)_{c}″ (III).

Very preferably, R₇ and R₈ independently represent a hydrogen atom, a methyl group, a 2-hydroxyethyl group, a 2-alkenyl group, a 2-aminoethyl group or a group of formula (III).

If the radical f represents the number 1 and the radical h represents the number 0, the organic silicon compound contains the grouping [NR₇-(A′)] but not the grouping —[NR₈-(A′″)].. If the radical R₇ now stands for a grouping of the formula (III), the anhydrous carrier medium contains an organic silicone compound with 3 reactive silane groups.

In a further preferred embodiment, the anhydrous carrier medium of the two-component system contains at least one organic silicon compound of formula (II)

(R₅O)_{c}(R₆)_{d}Si-(A)ₑ-[NR₇-(A′)]_{f}—[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently stand for a linear, divalent C₁-C₆-alkylene group and
R₇ represents a hydrogen atom, a methyl group, a 2-hydroxyethyl group, a 2-alkenyl group, a 2-aminoethyl group or a group of formula (III).

In a further preferred embodiment, the anhydrous carrier medium of the two-component system contains at least one organic silicon compound of formula (II), where
e and f both stand for the number 1,
g and h both stand for the number 0,
A and A′ independently of one another represent a methylene group(—CH₂—), an ethylene group (—CH₂—CH₂—) or a propylene group (—CH₂—CH₂—CH₂), and
R₇ represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem are

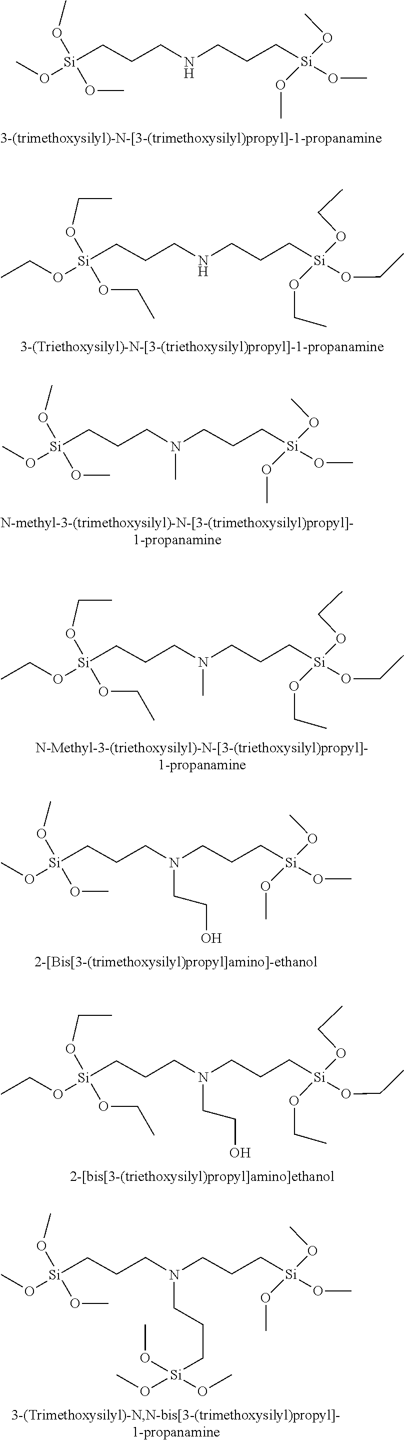

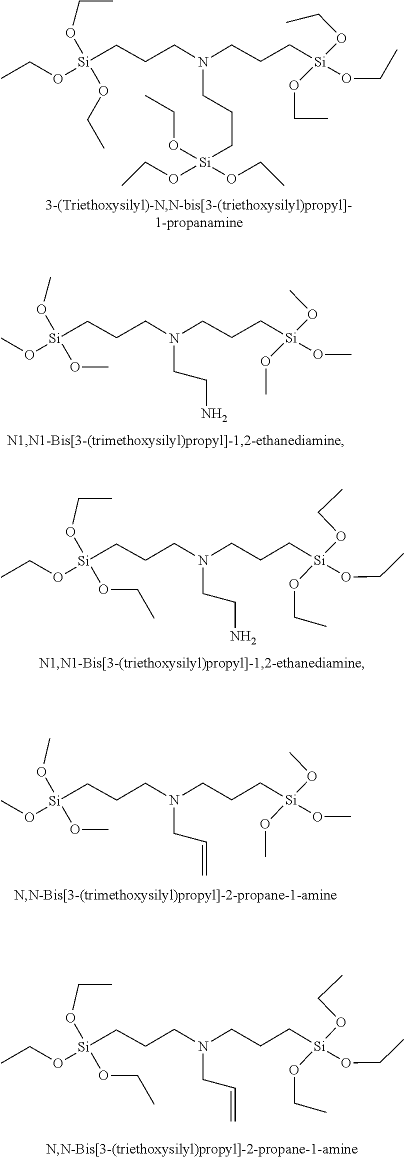

The aforementioned organic silicon compounds of formula (II) are commercially available.

Bis(trimethoxysilylpropyl)amine with the CAS number 82985-35- 1 can be purchased from Sigma-Aldrich®.

Bis[3-(triethoxysilyl)propyl]amine, also designated as 3-(Triethoxysilyl)-N-[3-(triethoxysilyl)propyl]-1-propanamine, with the CAS number 13497-18-2 is, for example, commercially available from Sigma-Aldrich® or can be commercially obtained from Evonik® under the product name Dynasylan® 1122.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich® or Fluorochem®.

3-(triethoxysilyl)-N,N-bis[3-(triethoxysilyl)propyl]-1-propanamine with the CAS number 18784-74-2 can be purchased for example from Fluorochem® or Sigma-Aldrich®.

It has also proved advantageous if the product for the treatment of a keratinous material contains at least one organic silicon compound of the formula (IV)

R₉Si(OR₁₀)ₖ(R₁₁)ₘ (IV).

The anhydrous carrier medium thus also contains an organic silicon compound of the formula (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be called a silane of the alkyl-alkoxy-silane or alkyl-hydroxy-silane type,

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further preferred embodiment, the anhydrous carrier medium contains, in addition to the organic silicon compound(s) of formula (I), at least one more organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a similarly preferred embodiment, the anhydrous carrier medium of the two-component system contains, in addition to the organic silicon compounds of formula (II), at least one more organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further preferred embodiment, the anhydrous carrier medium of the two-component system contains, in addition to the organic silicon compounds of the formula (I) and (II), at least one more organic silicon compound of the formula (IV).

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In the organic silicon compounds of formula (IV), the radical R₉ represents a C₁-C₁₂ alkyl group. This C₁-C₁₂ alkyl group is saturated and can be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R₁₀ represents a hydrogen atom or a C₁-C₆ alkyl group. Especially preferably, R₁₀ stands for a methyl group or an ethyl group.

In the organic silicon compounds of formula (IV), the radical R₁₁ represents a C₁-C₆ alkyl group. Especially preferably, R₁₁ stands for a methyl group or an ethyl group.

Furthermore k stands for a whole number from 1 to 3, and m stands for the whole number 3−k. If k stands for the number 3, then m is equal to 0. If k stands for the number 2, then m is equal to 1. If k stands for the number 1, then m is equal to 2.

It has proved to be very advantageous that the anhydrous carrier medium contains at least one organic silicon compound of formula (IV) in which the radical k represents the number 3. In this case the residue m stands for the number 0.

Organic silicon compounds of the formula (IV) which are well suited for solving the problem are

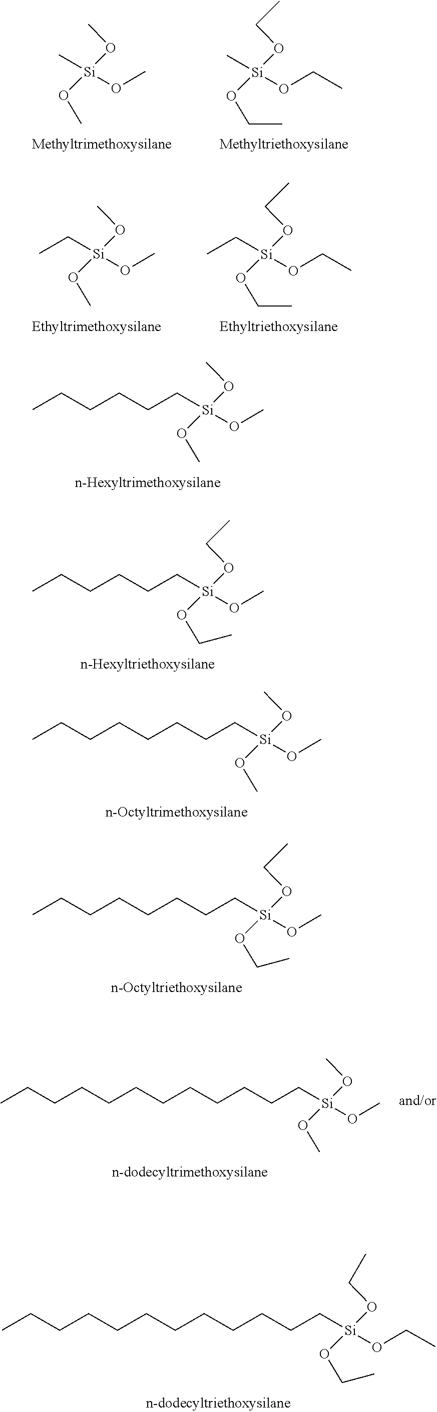

as well as propyltrimethoxysilane, propyltriethoxysilane, octadecyltrimethoxysilane and/or octadecyltriethoxysilane.

The organic silicon compounds described above are reactive compounds.

It has been observed that it is possible to obtain particularly sturdy and uniform films on the keratinous material, even if the anhydrous carrier medium contained two structurally different organic silicon compounds.

In a further preferred embodiment, an anhydrous carrier medium is exemplified in that it contains at least one organic silicon compound of formula (I) and at least one organic silicon compound of formula (IV).

In an explicitly specifically preferred embodiment, an anhydrous carrier medium exemplified in that it contains at least one organic silicon compound of the formula (I), which is selected from the group including (3-Aminopropyl)triethoxysilane and (3-Aminopropyl)trimethoxysilane, and additionally contains at least one organic silicon compound of the formula (IV), which is selected from the group including methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In a further preferred embodiment, an anhydrous carrier medium is exemplified in that the anhydrous carrier medium—based on the total weight of the anhydrous carrier medium contains:
from about 0.5 to about 5% by weight of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 10.0 weight % of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

In the case of organic silicon components with at least one hydrolysable group, the addition of small quantities of water already leads to hydrolysis. The hydrolysis products and/or organic silicon compounds with at least one hydroxy group can react with each other in a condensation reaction. For this reason, the anhydrous carrier medium can contain organosilicon compounds with at least one hydrolysable group as well as their hydrolysis and/or condensation products. When using organosilicon compounds with at least one hydroxyl group, the anhydrous carrier medium can contain the organic silicon compounds with at least one hydroxyl group as well as their condensation products.

A condensation product is understood to mean a product that is created due to the reaction of at least two organic silicon compounds each with at least one hydroxyl group or hydrolysable group per molecule on splitting of water and/or splitting of an alkanol. The condensation products can, for example, be dimers, or even trimers or oligomers, where in the condensation products are always in balance with the monomers. Depending on the water quantity added or consumed in the hydrolysis, the balance shifts from monomeric organic silicon compounds to condensation product.

In the context of the present disclosure, specifications in weight %—unless otherwise specified—are always in relation to the total weight of the anhydrous carrier medium. The details in weight % are in relation to the total weight of the aqueous phase, when applicable.

The second component of the two-component system of the present disclosure is an aqueous phase. This second component necessarily contains water and an alkali metal hydroxide. The alkali metal hydroxide effects the plasticity, i.e. straightening of the hair.

Various options for shaping of the hair are described in the state of the art. A permanent deformation of keratinous fibers is usually implemented such that one mechanically shapes the fiber and the shape is fixed by employing suitable resources. Before and/or after this shaping, one treats the fiber by employing a keratin-reducing preparation. After a rinsing procedure, the fiber is then treated in the fixing step by employing an oxidizer preparation, rinsed and freed of the shaping resources (rollers, papillotes) after or during the fixing step. If a mercaptan, such as ammonium thioglycolate, is used as the keratin-reducing component, it splits a part of the disulfide bridges of the keratin molecule to SH groups, such that the keratin fiber is softened. At the time of the later oxidative fixing, disulfide bridges are again linked in the hair keratin, such that the keratin structure is fixed in the specified shape. A known alternative is to use sulfite instead of the mercaptans for hair shaping. By employing hydrogen sulfite solutions and/or sulfite solutions and/or disulfite solutions, disulfite bridges of the keratin are split in a sulfitolysis according to the equation R—S—S—R+HSO₃⁻R—SH+R—S—SO₃⁻ and thus achieving a softening of the keratin fiber. Reducing agents containing hydrogen sulfite, sulfite or disulfite do not have the strong intrinsic odor of the mercaptan-containing agent. The splitting can be canceled, as described previously, in a fixing step by employing an oxidizing agent with the formation of new disulfite bridges.

The described method for permanent shaping places extraordinary stress on the hair. If the hair straightening to be effected is not supposed to be very permanent, it is also possible to allow an alkaline solution on the hair. By using mechanical stress, the hair can then be straightened after and/or during the effect of the alkaline solution.

In the present disclosure, the alkali metal hydroxide in the aqueous phase of the two-component system, after blending of aqueous phase and anhydrous carrier medium, leads to the two-component system acquiring an alkaline pH value and the two-component system can be used for hair shaping, especially straightening.

The anhydrous carrier medium necessarily contains three components as components of the anhydrous carrier medium: at least one branched or linear C8-C30 alkane, at least one branched or linear C10-C30 fatty alcohol, and at least one branched or linear monovalent C2-C8 alcohol. In the course of the work leading to the present disclosure, it turned out that it is advantageous for achieving a particularly good care effect, if the three components together with the organic silicon compounds, such as 3-(Triethoxysilyl)-N-[3-(triethoxysilyl)propyl]-1-propanamine, i.e. A bis(triethoxysilylpropyl)amine, were combined with the aqueous phase in the anhydrous carrier medium. By excluding water in the anhydrous carrier medium, the organic silicon compound is protected from premature hydrolysis and, only if needed, is converted to a hair care emulsion by mixing with a water phase, which activates the organic silicon compound by employing a hydrolysis reaction just before the application. It was surprisingly found that the combination of bis(triethoxysilylpropyl) amine and the three components essentially contained in the anhydrous carrier medium raised the cosmetic acceptance. The hair is soft, the combability increases distinctly and the hair surface is, particularly in the case of chemically treated hair, more hydrophobic. At the same time, a gentle straightening of the hair is achieved.

According to preferred embodiments of the present disclosure, the alkane in the two-component system is about a C10-C24 alkane, more preferably about a C12-C18 alkane and even more preferably about a C14-C16 alkane. It is preferred that the anhydrous carrier medium contain the alkane in a quantity of from about 1 to about 50 weight %, preferably from about 2 to about 45 weight %, more preferably from about 3 to about 40 weight %, even more preferably from about 4 to about 35 weight %, in relation to the total weight of the anhydrous carrier medium. The designation Cx to Cy always means that the hydrocarbon chain features x to y carbon atoms.

In a specifically preferred embodiment of the present disclosure, the anhydrous carrier is an anhydrous concentrate. In the embodiment, the quantity of alkane in the anhydrous carrier is from about 5 to about 90 weight %, preferably from about 10 to about 70 weight %, more preferably from about 15 to about 50 weight %.

According to a further preferred embodiment of the present disclosure, the fatty alcohol is about a C12-C24 fatty alcohol, preferably about a C14-C18 fatty alcohol, wherein the fatty alcohol is contained in the anhydrous carrier medium preferably in a quantity from about 5 to about 50 weight %, more preferably from about 6 to about 45 weight %, even more preferably from about 7 to about 40 weight %, most preferably from about 8 to about 35 weight %, in relation to the total weight of the anhydrous carrier medium.

According to a further preferred embodiment of the present disclosure, the alcohol is a C3-C6 alcohol, preferably a C4-C5 alcohol, wherein the alcohol is contained in the anhydrous carrier medium preferably in a quantity from about 4 to about 50 weight %, preferably from about 6 to about 45 weight %, more preferably from about 8 to about 40 weight %, even more preferably from about 10 to about 30 weight %, in relation to the total weight of the anhydrous carrier medium. A monohydric alcohol is to be understood as an alcohol with only one OH function.

The two-component system comprises, according to the preferred embodiments, emulsifiers, so that their components, i.e. the anhydrous carrier medium and the aqueous phase, can be homogenized and form a homogeneous phase at the time of manufacture of the ready-to-use cosmetic product. According to this preferred embodiment, the emulsifier or emulsifiers are contained in the aqueous phase or in the anhydrous carrier medium, but preferably in the aqueous phase.

A preferably used emulsifier is an alkyltrimonium compound with one or multiple about C8-C22, more preferably about C10-C18, even more preferably about C12-C16 alkyl groups. An alkyltrimonium compound is a nitrogen-containing compound, which is cationic and bears at least one, preferably multiple organic radicals. The number of the organic radicals plus hydrogen always yields the number four.

A further preferred emulsifier is a fatty alcohol ethoxylate, in which the fatty alcohol part of the fatty alcohol ethoxylate features an alkyl chain length of about C4-C30, preferably about C6-C25, more preferably about C8-C20 and/or in which the number of ethoxy groups in the fatty alcohol ethoxylate is from 2 to about 120, preferably from 4 to about 100, more preferably from about 6 to about 80, even more preferably from about 8 to about 60, most preferably from about 10 to about 40.

Furthermore, a preferred emulsifier is a fatty alcohol sulfate with a chain length of about C8-C22, preferably of about C10-C20, more preferably of about C12-C18.

According to further preferred embodiments, the emulsifiers are contained in defined quantities either in the anhydrous carrier medium or in the aqueous phase. A preferred two-component system contains the alkyltrimonium compound in the aqueous phase in a quantity from about 0.1 to about 5 weight %, preferably from about 0.5 to about 4 weight %, more preferably from about 1 to about 3 weight %, in relation to the total weight of the aqueous phase, or the alkyltrimonium compound is in the anhydrous carrier medium in a quantity from about 0.1 to about 5 weight %, preferably from about 0.5 to about 4 weight %, more preferably from about 1 to about 3 weight %, in relation to the total weight of the anhydrous carrier medium.

According to a further preferred embodiment, the aqueous phase contains the fatty alcohol ethoxylate in a quantity from about 0.1 to about 10 weight %, preferably from about 0.25 to about 7.5 weight %, more preferably from about 0.5 to about 5 weight %, in relation to the total weight of the aqueous phase, and the anhydrous carrier medium contains the fatty alcohol ethoxylate in a quantity from about 0.1 to about 10 weight %, preferably from about 0.25 to about 7.5 weight %, more preferably from about 0.5 to about 5 weight %, in relation to the total weight of the anhydrous carrier medium.

Furthermore, according to preferred embodiments, the aqueous phase contains the fatty alcohol sulfate in a quantity from about 0.1 to about 10 weight %, preferably from about 0.25 to about 7.5 weight %, more preferably from about 0.5 to about 5 weight %, in relation to the total weight of the aqueous phase, or the anhydrous carrier medium contains fatty alcohol sulfate in a quantity from about 0.1 to about 10 weight %, preferably from about 0.25 to about 7.5 weight %, preferably from about 0.5 to about 5 weight %, in relation to the total weight of the anhydrous carrier medium.

According to a preferred embodiment of the present disclosure, the alkali metal hydroxide in the two-component system is lithium hydroxide, sodium hydroxide, potassium hydroxide or any arbitrary mixture thereof, wherein the aqueous phase contains the alkali metal hydroxide preferably in a quantity from about 0.1 to about 5 weight %, preferably from about 0.2 to about 4 weight % and more preferably from about 0.3 to about 3 weight %, in relation to the weight of the aqueous phase.

According to a preferred embodiment of the present disclosure, the weight proportion of the anhydrous carrier medium to the aqueous phase in the two-component system is from about 1 to about 10 up to from about 10 to about 1, preferably from about 5 to about 1 up to from about 1 to about 5, more preferably from about 2 to about 1 up to from about 1 to about 2.

All physiologically compatible counter ions can be used as counter ions of the present components that are present as salts.

The aqueous phase or the anhydrous carrier medium can comprise other additional ingredients in further preferred embodiments of the present disclosure. The following ingredients are optional and contained in addition to the above described components in the two-component system.

In another preferred two-component system, the anhydrous carrier medium or the aqueous phase contains a cationic surfactant, wherein the cationic surfactant has one of the following formulas:

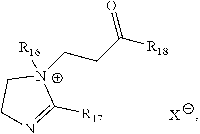

wherein
R₁₆ represents a C1-C6 alkyl group
R₁₇, R₁₈ independently represent about a C7-C27-alkyl group, preferably about a C10-C22-alkyl group and
X− stands for a physiologically compatible anion,
or, in which the cationic surfactant has one of the following formulas:

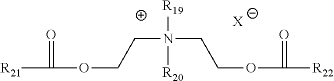

wherein
R₁₉, R₂₀, independently represent a C1-C6-Alkyl group and a C2-C6-hydroxyalkyl group,
R₂₁, R₂₂ independently represent about a C7-C27-alkyl group, preferably about a C10-C22-alkyl group and
X− stands for a physiologically compatible anion,

Or, in which the cationic surfactant has one of the following formulae:

NR₂₃R₂₄R₂₅,

wherein
R₂₃, R₂₄ independently represent a C1-C6-alkyl group, a C2-C6-alkenyl group or a C2-C6-hydroxyalkyl group, and
R₂₅ represents about a C8-C28-alkyl group, preferably about a C10-C22-alkyl group.

The cationic surfactants of the formula NR₂₃R₂₄R₂₅ are amino derivatives that are called pseudo quats. The organic radicals R₂₃, R₂₄ and R₂₅ are directly connected to the nitrogen atom in that case. In the acidic pH range, these are cationized i.e. the nitrogen atom is then protonized. The physiologically compatible counter-ions are then available as counter ions. Steamidopropyl dimethylamine is especially preferred among the cationic surfactants.

In the present disclosure, the surfactants support the emulsification of the aqueous phase and the anhydrous carrier medium or generally emulsification of water and oil phases. They are therefore optionally additionally contained in the two-component system.

Other components of the hair treatment product shall be described hereafter, which can be optionally contained in the anhydrous carrier medium or the aqueous phase, besides the above-described mandatory ingredients.

According to further preferred embodiments, the anhydrous carrier medium or the aqueous phase further contains a skin moisturizer or other care agent that is selected from the group including glycerin, urea, hyaluronic acid, silanol ester of the hyaluronic acid, panthenol, taurine, ceramides, phytosterols, aloe vera extracts, creatine, creatinine, sodium hyaluronate, polysaccharides, biosaccharides gum-1, cucumber extracts, butylene glycol, propylene glycol, methyl propane diol, ethylhexylglycerin, sorbitol, amino acids, wherein glycine, glycine soy, histidine, tyrosine or tryptophan are especially preferred amino acids, amino acid derivatives, natural betaine compounds, pyrrolidone carboxylic acid or a salt of pyrrolidone carboxylic acid, lactic acid, lactates, especially sodium lactate, and/or ethylhexyloxyglycerin. Particularly, the selection of these skin moisturizers enhances the care character of the anhydrous carrier medium.

According to a further preferred embodiment, the anhydrous carrier medium or the aqueous phase contains multiple emulsifiers or multiple surfactants. It is particularly preferred that the anhydrous carrier medium or the aqueous phase contains two structurally mutually different surfactants/emulsifiers, wherein preferably the anhydrous carrier medium or the aqueous phase contains two structurally mutually different cationic emulsifiers/surfactants, two mutually different anionic emulsifiers/surfactants, a cationic surfactant/emulsifier and a non-ionic surfactant/emulsifier, or an anionic surfactant/emulsifier and a non-ionic surfactant/emulsifier.

According to a preferred embodiment of the present disclosure, the cationic surfactant comprises a hydrophobic head group with a cationic charge and one or two hydrophobic end parts, wherein the hydrophobic end part or the hydrophobic end parts represent straight chained or branched, saturated or mono- or poly-unsaturated alkyl groups, which preferably feature a chain length of C6 to C30, preferably C8 to C26, and especially preferably C10 to C22. According to a further preferred embodiment, the cationic surfactant features an ester function, an ether function, a ketone function, an alcohol function or an amide function.

According to a preferred embodiment of the present disclosure, the anhydrous carrier medium or the aqueous phase contains, as another component, a further non-ionic surfactant, which preferably comprises a non-ionic surfactant selected from the group including the following:
Alkyl glucamide, comprising a saturated or unsaturated, branched or unbranched about C₆ to about C₂₂, preferably about C₁₀ to about C₁₈, more preferably about C₁₂ to about C₁₆ alkyl group,
Alkyl fructoside, comprising a saturated or unsaturated, branched or unbranched about C₆ to about C₂₂, preferably about C₁₀ to about C₁₈, more preferably about C₁₂ to about C₁₆ alkyl group,
Alkyl glucoside, comprising a saturated or unsaturated, branched or unbranched about C₆ to about C₂₂, preferably about C₁₀ to about C₁₈, more preferably about C₁₂ to about C₁₆ alkyl group, and
Alkyl alcoholalkoxylate of the formula R₁₀(OR₁₁)ₘOH, in which R₁₀ represent a linear or branched about C₆ to about C₂₂, preferably about C₁₀ to about C₁₈, more preferably about C₁₂ to about C₁₆ alkyl group, R₁₁ represents a C₂ to C₄, preferably a C₂ alkyl group, and m represents 1 to about 10, preferably 2 to 6, more preferably 2 to 6.

According to preferred embodiments of the present disclosure, the anhydrous carrier medium or the aqueous phase contains one or more further anionic surfactants as a component, which is preferably selected from the group including:
linear alpha olefin sulfonates with about 8 to about 24, preferably about 12 to about 22, more preferably about 16 to about 18 C atoms,
straight chained or branched, saturated or mono- or poly-unsaturated alkyl carboxylic acids with about 8 to about 24, preferably about 12 to about 22, more preferably about 16 to about 18 C atoms,
straight chained or branched, saturated or mono- or poly-unsaturated alkyl sulfonates with about 8 to about 24, preferably about 12 to about 22, more preferably about 16 to about 18 C atoms,
Alkyl isethionate, whose alkyl group is selected from a branched or unbranched about C₆ to about C₂₂, preferably about C₁₀ to about C₁₈, more preferably about C₁₂ to about C₁₆ alkyl group, especially sodium cocoyl isethionate,
Alkyl glycoside carboxylic acids, whose alkyl group is selected from a branched or unbranched about C₆ to about C₂₂, preferably about C₁₀ to about C₁₈, more preferably about C₁₂ to about C₁₆ alkyl group,
Alkyl sulfosuccinates, whose two alkyl groups are selected from the same or different branched or unbranched C₂ to about C₁₂, preferably C₄ to about C₁₀, more preferably about C₆ to about C₈ alkyl groups,
Alkyl taurates, whose alkyl group is selected from a branched or unbranched about C₆ to about C₂₂, preferably about C₁₀ to about C₁₈, more preferably about C₁₂ to about C₁₆ alkyl group,
Alkylsarcosinates, whose alkyl group is selected from a branched or unbranched about C₆ to about C₂₂, preferably about C₁₀ to about C₁₈, more preferably about C₁₂ to about C₁₆ alkyl group,
Sulfonates of unsaturated fatty acids with about 8 to about 24, preferably about 12 to about 22, more preferably about 16 to about 18 C atoms and 1 to about 6 double bonds,
   Wherein the counter ion of the anionic surfactant is an alkali or earth alkali metal ion or a protonized triethanol amine or the ammonium ion.

The aqueous phase very particularly preferably contains a surfactant mix of anionic and amphoteric/zwitterionic surfactants sodium lauryl ether sulfate (INCI: Sodium laureth sulfate) and specifically preferred sodium lauryl ether sulfate with 2 ethylene oxide units.

Amphoteric surfactants, which are also designated zwitterionic surfactants, are those surface-active compounds which carry at least one quaternary ammonium group and at least one —COO⁽⁻⁾— or —SO₃⁽⁻⁾ group in the molecule. Amphoteric/zwitterionic surfactants are also those surface-active compounds which, apart from about a C₈-C₂₄ alkyl or acyl group, contain at least one free amino group and at least one —COOH or —SO₃H group in the molecule and are capable of forming internal salts.

According to a preferred embodiment of the present disclosure, the anhydrous carrier medium or the aqueous phase contains at least one amphoteric surfactant as a further component. Preferably, the amphoteric surfactants in the anhydrous carrier medium are selected from the group including:
Alkyl betaine, comprising a saturated or unsaturated, branched or unbranched about C₆ to about C₂₂, preferably about C₁₀ to about C₁₈, more preferably about C₁₂ to about C₁₆ alkyl group,
Alkyl ampho diacetate or alkyl ampho diacetate, comprising a saturated or unsaturated, branched or unbranched about C₆ to about C₂₂, preferably about C₁₀ to about C₁₈, more preferably about C₁₂ to about C₁₆ alkyl group, with an alkali or earth alkali metal counter ion, and
Alkyl amide propyl betaine, comprising a saturated or unsaturated, branched or unbranched about C₆ to about C₂₂, preferably about C₁₀ to about C₁₈, more preferably about C₁₂ to about C₁₆ alkyl group,

The especially suitable amphoteric/zwitterionic surfactants also include the surfactants known under the INCI designation cocamidopropylbetaine and di-sodium cocoamphodiacetate.

The anhydrous carrier medium can especially be used for the manufacture of an agent for the straightening of a keratinous material, an agent for the care of a keratinous material, an agent for care and cleaning of a keratinous material, and/or of an agent for coloring a keratinous material.

It can be preferable that the anhydrous carrier medium or the aqueous phase further comprises about 0.001 to about 20 weight % of at least one quaternary compound for the treatment of a keratinous material. This applies particularly to anhydrous carrier mediums or aqueous phases, which are used for the manufacture of an agent for the care of a keratinous material or for care and cleaning of a keratinous material.

It is preferred that the at least one quaternary compound be selected from at least one of the groups including:

i) the monoalkyl quats and/or
ii) the ester quats and/or
iii) the quaternary imidazoline of the formula (Tkat2).

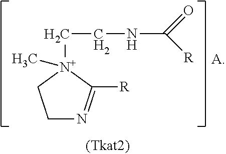

In which the radical R independently stands for a saturated or unsaturated, linear or branched hydrocarbon radical with a chain length from about 8 to about 30 carbon atoms and A stands for a physiologically compatible anion, and/or

iv) the amidoamines and/or cationized amidoamines and/or
v) poly(methacryloyloxyethyltrimethyl ammonium compounds) and/or
vi) quaternized cellulose derivatives, especially polyquaternium 10, polyquaternium-24, polyquaternium-27, polyquaternium-67, polyquaternium-72 and/or
vii) Cationic alkyl polyglycosides and/or
viii) Cationized honey and/or
ix) Cationic guar derivatives and/or
x) Chitosan and/or
xi) polymeric dimethyldiallylammonium salts and their copolymers with esters and amides of acrylic acid and methacrylic acid, mainly polyquaternium-7 and/or
xii) Copolymers of the vinyl pyrrolidone with quaternized derivatives of the dialkylaminoalkylacrylates and methacrylates, especially polyquaternium-11 and/or
xiii) vinylpyrrolidone-vinylimidazoliummethochloride-copolymers, especially polyquaternium-16 and/or
xiv) Quaternized polyvinyl alcohol and/or

### xv) Polyquaternium-74

and mixtures thereof.

It is especially preferred that the anhydrous carrier medium or the aqueous phase contain a cationic homopolymer, which falls under the INCI designation polyquaternium-37, as quaternary compounds.

It can be preferable that the anhydrous carrier medium or the aqueous phase further comprise one solidifying compound, preferably selected from the group including waxes, synthetic polymers and mixtures thereof.

In order to cope with the different requirements for cosmetic agents that are used for the manufacture of a product for the treatment of a keratinous material or for temporary shaping of a keratinous material (=styling agent), a multitude of synthetic polymers has been developed as solidifying compounds, which can be used in the product for the treatment of a keratinous material. Waxes are used alternatively or additionally as solidifying compounds. Ideally, the polymers and/or waxes when used on the keratinous material leave a polymer film that lends a strong hold to the hairstyle on the one hand, is however sufficiently flexible on the other, so that it does not break on being stressed.

The synthetic polymers can be divided in cationic, anionic, nonionic and amphoteric solidifying polymers.

Suitable synthetic polymers comprise, for example, polymers with the following INCI designation. Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxy esters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis- Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Cross polymer, Diphenyl Amodimethicone, Ethyl Ester of PVM/MA Copolymer, Hydrolyzed Wheat Protein/PVP Cross polymer, Isobutylene/Ethylmaleimide/Hydroxy ethylmaleimide Copolymer, Isobutylene/MA Copolymer, Isobutyl methacrylate/Bis-Hydroxypropyl Dimethicone Acrylate Copolymer, Isopropyl Ester of PVM/MA Copolymer, Lauryl Acrylate Cross polymer, Lauryl Methacrylate/Glycol Dimethacrylate Cross polymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Cross polymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilyl carbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethyl methacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers, such as hydroxypropylcellulose, hydroxyethylcellulose and methylhydroxypropylcellulose are also suitable.

Also Homopolyacrylic acid (INCI: Carbomer), which is commercially available under the name Carbopol® in different versions, is suitable as a solidifying compound.

Preferably, the solidifying compound comprises a vinylpyrrolidone-containing polymer. Particularly preferably, the solidifying compound comprises a polymer selected from the group including polyvinylpyrrolidone (PVP), vinylpyrrolidone-vinylacetate-copolymer (VP/VA copolymer), vinyl caprolactam/VP/dimethylaminoethyl methacrylate copolymer (INCI), VP/DMAPA acrylates copolymer (INCI) and mixtures thereof.

A similarly preferred solidifying compound is octylacrylamide/acrylates/butylaminoethyl methacrylate copolymer (INCI), which is sold under the name “Amphomer®” by Akzo Nobel®.

Accordingly, it is particularly preferable, that the solidifying compound comprises a synthetic polymer selected from the group including polyvinylpyrrolidone (PVP), vinylpyrrolidone-vinylacetate-copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI) and mixtures thereof.

The cosmetic products can, additionally or alternatively to a synthetic polymer, contain at least one natural or synthetic wax, which has a melting point above about 37° C., as a solidifying compound.

As natural or synthetic waxes, solid paraffin or isoparaffins, vegetable waxes such candelila wax, carnauba wax, esparto grass wax, Japan wax, cork wax, sugar cane wax, ouricury wax, montan wax, sunflower wax, fruit waxes and animal waxes such as beeswaxes and other insect waxes, whale wax, shellac wax, wool wax and brushing grease, furthermore mineral waxes such as Ceresin and Ozokerite or the petrochemical waxes such as petrolatum, paraffin wax, microwaxes from polyethylene or polypropylene and polyethylene glycol waxes can be used. It can be advantageous to use hydrated or hardened waxes. Furthermore, chemically modified waxes, especially resin waxes, for example montan ester waxes, sasol waxes and hydrated jojoba waxes can also be used.

Furthermore, the triglycerides of saturated and unsaturated hydroxylated C about 16-30 fatty acids, such as hardened triglyceride fats (hydrated palm oil, hydrated coconut oil, hydrated castor oil), glyceryl tribehenate or glyceryl tri-12 hydroxy stearate, are suitable.

The wax components can also be selected from the group of esters of saturated, unbranched alkane carboxylic acids of a chain length of about 22 to about 24 C atoms and saturated, unbranched alcohols of a chain length of about 22 to about 24 C atoms, if the wax components or the totality of the wax components are solid at room temperature. Silicone waxes, such as stearyltrimethylsilane/stearyl alcohol can also be advantageous.

Natural, chemically modified and synthetic waxes can be used alone or in combination. This should, however, not include alkanes that are necessarily contained in the anhydrous carrier medium as contemplated herein. Thus, even several waxes can be used. Moreover, a series of wax mixes, possibly in mixture with other additives, is also commercially available. Examples of usable mixtures are the ones available under the names “Special wax 7686 OE’ (a mixture of cetyl palmitate, bees wax, micro crystalline wax and polyethylene with a melting range of about 73-75° C., manufacturer: Kahl® & Co), Plywax® GP 200 (a mixture of stearyl alcohol and polyethylene glycol stearate with melting point of about 47-51° C.; manufacturer: Croda®) and “Weichceresin® FL 400” (a vaseline/vaseline oil/wax mixture with a melting point of about 50-54° C.; manufacturer: Parafluid Mineralölgesellschaft).

The waxes selected from Carnauba wax (INCI: Copernicia Cerifera Cera) bees wax (INCI: Beeswax), Petrolatum (INCI), micro crystalline wax and especially mixtures therefrom are preferred.

Preferred mixtures comprise the combination of Carnauba wax (INCI: Copernicia Cerifera Cera), Petrolatum and micro crystalline wax or the combination of bees wax (INCI: Beeswax) and Petrolatum.

The wax or the wax components should be solid at about 25° C. and should melt in the range of >about 37° C.

The anhydrous carrier medium or the aqueous phase contains the solidifying compound preferably in a total quantity of from about 0.5 to about 50 weight %, preferably from about 1 to about 40 weight %, more preferred from about 1.5 to about 30 weight %, even more preferred from about 2 to about 25 weight %, based on the total weight of the cosmetic composition.

Other suitable ingredients comprise non-ionic polymers, anionic polymers, (other) cationic polymers, waxes, protein hydrolysates, amino acids, oligopetides, vitamins, pro-vitamins, vitamin precursors, betaines, biochinones, purine (derivatives), plant extracts, silicones, ester oils, UV light filters, structuring agents, thickening agents, electrolytes, pH adjusters, swelling agents, dyes, anti-dandruff agents, complexing agents, opacifiers, pearlizing agents, pigments, stabilizers, propellants, anti-oxidants, perfume oils and/or preservatives.

Another object of the present application is the use of the two-component system for the straightening of a keratinous material, for care of a keratinous material and/or for the care and cleaning of a keratinous material.

Regarding further preferred embodiments of the use, what was said about the anhydrous carrier mediums is applicable mutatis mutandis.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A two-component system for the straightening and care of keratinous material, separately comprising
an anhydrous carrier medium as a first component, the anhydrous carrier medium comprising:
a) at least one branched or linear C8-C30 alkane,
b) at least one branched or linear C10-C30 fatty alcohol, and
c) at least one branched or linear monovalent C2-C8 alcohol, and
an aqueous phase as a second component, the aqueous phase comprising water and an alkali metal hydroxide.

2. The two-component system as claimed in to claim 1, wherein the anhydrous carrier medium further comprises at least one organic silicon compound that represents a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, divalent C₁-C₆-alkylene group,
R₃, R₄ independently represent a methyl group or an ethyl group,
a stands for the number 3 and
b stands for the number 0 and
wherein in the organic silicon compound of formula (II)
(R₅O)_{c}(R₆)_{d}Si-(A)ₑ-[NR₇-(A′)]_{f}—[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} independently represent a C₁-C₆ alkyl group,
A, A′, A″, A′″ and A″″ independently represent a linear or branched, divalent C₁-C₂₀-alkylene group,
R₇ and R₈ independently represent a hydrogen atom, a C₁-C₆-alkyl group, a hydroxy-C₁-C₆-alkyl group, a C₂-C₆-alkenyl group, an amino-C₁-C₆-alkyl group or a grouping of the formula (III)
-(A″″)—Si(R₆″)_{d″}(OR₅″)_{c″} (III),
wherein:
c stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
with the proviso that at least one of the radicals from e, f, g and h is different from 0.

3. The two-component system as claimed in claim 2,
wherein
the two-component system for the treatment of a keratinous material comprises the at least one organic silicon compound of the formula (I), which is selected from the group of
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-dimethylaminoethyl)trimethoxysilane and
(2-Dimethylaminoethyl)triethoxysilane.

4. The two-component system as claimed in claim 2, wherein
the two-component system comprises the at least one organic silicon compound of the formula (II), which is selected from the group of:
3-(trimethoxysilyl)-N-[3-(trimethoxysilyl)propyl]-1-propanamine,
3-(Triethoxysilyl)-N-[3-(triethoxysilyl)propyl]-1-propanamine,
N-methyl-3-(trimethoxysilyl)-N-[3-(trimethoxysilyl)propyl]-1-propanamine,
N-Methyl-3-(triethoxysilyl)-N-[3-(triethoxysilyl)propyl]-1-propanamine,
2-[Bis[3-(trimethoxysilyl)propyl]amino]-ethanol,
2-[bis[3-(triethoxysilyl)propyl]amino]ethanol,
3-(Trimethoxysilyl)-N,N-bis[3-(trimethoxysilyl)propyl]-1-propanamine,
3-(Triethoxysilyl)-N,N-bis[3-(triethoxysilyl)propyl]-1-propanamine,
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl) propyl]-2-propene-1-amine, and/or
N,N-bis[3-(triethoxysilyl)propyl]-2-propene-1-amine.

5. The two-component system as claimed in claim 1, wherein the two-component system comprises at least one organic silicon compound of the formula (IV)
R₉Si(OR₁₀)ₖ(R₁₁)ₘ (IV),
which is selected from the group of:
Methyltrimethoxysilane,
Methyltriethoxysilane,
Ethyltrimethoxysilane,
Ethyltriethoxysilane,
Propyltrimethoxysilane,
Propyltriethoxysilane,
Hexyltrimethoxysilane,
Hexyltriethoxysilane,
Octyltrimethoxysilane,
Octyltriethoxysilane,
Dodecyltrimethoxysilane,
Dodecyltriethoxysilane,
Octadecyltrimethoxysilane, and
Octadecyltriethoxysilane.

6. The two-component system as claimed in claim 2, wherein the anhydrous carrier medium comprises the organic silicon compound in a quantity of from about 0.01 to about 10 weight %, based on the total weight of the anhydrous carrier medium.

7. The two-component system as claimed in claim 1, wherein the alkane is a C10-C24 alkane, and wherein anhydrous carrier medium comprises the alkane in a quantity of from about 1 to about 50 weight %, in relation to a total weight of the anhydrous carrier medium, and wherein,
the fatty alcohol is a C12-C24 fatty alcohol, wherein the anhydrous carrier medium comprises the fatty alcohol in a quantity of from about 5 to about 50 weight %, in relation to the total weight of the anhydrous carrier medium, and wherein,
the alcohol is a C3-C6 alcohol, wherein the anhydrous carrier medium comprises the alcohol in a quantity of from about 4 to about 50 weight %, in relation to the total weight of the anhydrous carrier medium.

8. The two-component system as claimed in claim 1, wherein the aqueous phase or the anhydrous carrier medium comprises one or multiple emulsifiers, wherein the emulsifier is selected from the group of:
an alkyltrimonium compound with one or multiple C8-C22,
a fatty alcohol ethoxylate, in which the fatty alcohol part of the fatty alcohol ethoxylate features an alkyl chain length of C4-C30, and in which the number of ethoxy groups in the fatty alcohol ethoxylate is 2 to 120,
a fatty alcohol sulfate with a chain length of C8-C22,
and combinations thereof.

9. The two-component system as claimed in claim 8, wherein the aqueous phase comprises the alkyltrimonium compound in a quantity of from about 0.1 to about 5 weight %, in relation to the total weight of the aqueous phase.

10. The two-component system as claimed in claim 1, wherein the the alkali metal hydroxide is selected from the group of lithium hydroxide, sodium hydroxide, potassium hydroxide, and an arbitrary mixture thereof, and wherein the aqueous phase comprises the alkali metal hydroxide in a quantity of from about 0.1 to about 5 weight %, in relation to a weight of the aqueous phase.

11. The two-component system, as claimed in claim 1 wherein the weight proportion of the anhydrous carrier medium to the aqueous phase is in the range of from about 1 about 10 to from about 10 to about 1.

12. A method for straightening or caring for keratinous material, the method comprising:
applying a two-component system to the keratinous material, wherein the two-component system comprises:
an anhydrous medium as a first component, wherein the anhydrous medium comprises a branched or linear C8-C30 alkane, a branched or linear C10-C30 fatty alcohol, and a branched or linear monovalent C2-C8 alcohol, and
an aqueous phase as a second component, wherein the aqueous phase comprises water and an alkali metal hydroxide.

13. The two-component system as claimed in claim 2, wherein the anhydrous carrier medium comprises the organic silicon compound in a quantity of from about 0.1 to about 7 weight %, based on a total weight of the anhydrous carrier medium, and wherein the organic silicon compound of formula (I) is (3-Aminopropyl)triethoxysilane.

14. The two-component system as claimed in claim 8, wherein:
the anhydrous carrier medium comprises the alkyltrimonium compound in a quantity of from about 0.1 to about 5 weight %, in relation to the total weight of the anhydrous carrier medium.

15. The two-component system as claimed in claim 8, wherein:
the aqueous phase comprises the fatty alcohol ethoxylate in a quantity of from about 0.1 to about 10 weight %, in relation to the total weight of the aqueous phase.

16. The two-component system as claimed in claim 8, wherein:
the anhydrous carrier medium comprises the fatty alcohol ethoxylate in a quantity of from about 0.1 to about 10 weight %, in relation to the total weight of the anhydrous carrier medium.

17. The two-component system as claimed in claim 8, wherein:
the aqueous phase comprises the fatty alcohol sulfate in a quantity of from about 0.1 to about 10 weight %, in relation to the total weight of the aqueous phase.

18. The two-component system as claimed in claim 8, wherein:
the anhydrous carrier medium comprises the fatty alcohol sulfate in a quantity of from about 0.1 to about 10 weight %, in relation to the total weight of the anhydrous carrier medium.

19. The two-component system as claimed in claim 2, wherein the anhydrous carrier medium comprises the organic silicon compound in a quantity of from about 0.05 to about 8 weight %, based on a total weight of the anhydrous carrier medium.

20. The two-component system as claimed in claim 1, wherein:
the alkane is a C14-C16 alkane, and wherein anhydrous carrier medium comprises the alkane in a quantity of from about 4 to about 35 weight %, in relation to a total weight of the anhydrous carrier medium,
the fatty alcohol is a C14-C18 fatty alcohol, wherein the anhydrous carrier medium comprises the fatty alcohol in a quantity of from about 8 to about 35 weight %, in relation to the total weight of the anhydrous carrier medium, and
the alcohol is a C4-C5 alcohol, wherein the anhydrous carrier medium comprises the alcohol in a quantity of from about 10 to about 30 weight %, in relation to the total weight of the anhydrous carrier medium.
